(19) United States
(12) Patent Application Publication
(10) Pub. No.: US 2026/0115353 A1
(43) Pub. Date: Apr. 30, 2026

(54) HYDROLYZED COLLAGEN COMPOSITIONS WITH ANTIBIOTICS AND METHODS OF USING THE SAME

(71) Applicant: Rochal Technologies, LLC, San Antonio, TX (US)

(72) Inventors: Nina Bionda, Ithaca, NY (US); Larry E. Estlack, San Antonio, TX (US); Jonathan D. Velasco, San Antonio, TX (US); Ann Beal Salamone, Gainesville, FL (US); Rebecca E. McMahon, San Antonio, TX (US)

(73) Assignee: Rochal Technologies, LLC, San Antonio, TX (US)

(21) Appl. No.: 19/371,959

(22) Filed: Oct. 28, 2025

Related U.S. Application Data
(60) Provisional application No. 63/712,879, filed on Oct. 28, 2024.

(51) Int. Cl. A61L 26/00; C08L 89/06

(52) U.S. Cl. CPC A61L 26/0033; A61L 26/0066; C08L 89/06; A61L 2300/406; A61L 2400/06

(57) ABSTRACT

A hydrolyzed collagen-based composition is provided. The composition includes 25 wt % to 99.9995 wt % of hydrolyzed collagen; and 0.0005 wt % to 50 wt % of an antibiotic component, wherein the percentages are based on the percent solids of the composition. Methods of using the composition are also provided.

### CROSS-REFERENCE TO RELATED APPLICATIONS

This application claims priority under 35 U.S.C. § 119(e) to U.S. Provisional Application No. 63/712,879, filed on Oct. 28, 2024, the entire contents of which are incorporated herein by reference.

### FIELD OF INVENTION

A hydrolyzed collagen composition with biocidal activity is described, in particular a composition with hydrolyzed collagen and at least one antibiotic in order to prevent, reduce, or eliminate microbial infections.

### BACKGROUND

Hydrolyzed collagen is derived from acid, alkaline, or enzymatic hydrolysis of collagen, which reduces the molecular weight from about 300 kDa down to a molecular weight that is generally less than 12 kDa. Hydrolyzed collagen has high concentrations of oligomers containing hydroxyproline (hydrophilic), proline (hydrophobic), and glycine (hydrophilic), low content of sulfur-containing amino acids, and no tryptophan.

When dissolved in aqueous media, hydrolyzed collagen produces a low viscosity solution at lower concentrations but can form a water-soluble gel at higher concentrations (e.g., over 60 wt-%). When dried, this hydrolyzed collagen gel forms a brittle coating that is readily water soluble. Hydrolyzed collagen can be used in a variety of ways.

### SUMMARY OF THE INVENTION

A hydrolyzed collagen-based composition comprising 25 wt % to 99.9995 wt % of hydrolyzed collagen; and 0.0005 wt % to 50 wt % of an antibiotic component, wherein the percentages are based on the percent solids of the composition. The hydrolyzed collagen-based composition can have antimicrobial properties to mitigate or eliminate infection while maintaining or improving biocompatibility.

In some embodiments, the antibiotic component comprises at least one of a polyene antimycotic, an aminoglycoside antibiotic, a glycopeptide antibiotic, beta-lactam antibiotic, or a derivative thereof.

In some embodiments, the hydrolyzed collagen-based compositions described herein can be used to reduce or eliminate microbes in drugs and nutritional supplements.

In some embodiments, the hydrolyzed collagen-based compositions described herein can be used in personal care products.

In some embodiments, the hydrolyzed collagen-based compositions described herein can be used to treat surgical incision sites.

In some embodiments, the hydrolyzed collagen-based compositions described herein can be used to treat impaired soft tissue or hard tissue.

In some embodiments, the hydrolyzed collagen-based compositions described herein can be used to treat acute and chronic wounds, as well as, burn wounds.

In some embodiments, the hydrolyzed collagen-based compositions described herein can be used to reduce and/or eliminate Gram-positive and Gram-negative bacteria in or on wounds, tissues, surfaces and devices.

In some embodiments, the hydrolyzed collagen-based compositions described herein can be used to reduce and/or eliminate fungi in or on wounds, tissues, surfaces and devices.

In some embodiments, the hydrolyzed collagen-based compositions described herein can be used to reduce and/or eliminate yeast in or on wounds, tissues, surfaces and devices.

In some embodiments, the hydrolyzed collagen-based compositions described herein can be used to reduce and/or eliminate mold in or on wounds, tissues, surfaces and devices.

In some embodiments, the hydrolyzed collagen-based compositions described herein can be used to reduce and/or eliminate viruses in or on wounds, tissues, surfaces and devices.

In some embodiments, the hydrolyzed collagen-based compositions described herein can be used to facilitate tissue healing by incorporation of hydrolyzed collagen and antibiotics.

In some embodiments, the hydrolyzed collagen-based compositions described herein can include antibiotics-containing compositions that are non-cytotoxic to mammalian cells.

In some embodiments, the hydrolyzed collagen-based compositions described herein can be deposited on a surface of a device to reduce and/or eliminate microbes while providing biocompatibility.

These and other objectives and advantages of the hydrolyzed collagen-based compositions described herein, some of which are specifically described and others that are not, will become apparent from the detailed description and drawings that follow.

### DETAILED DESCRIPTION OF THE INVENTION

In this specification and the appended claims that follow, reference will be made to a number of terms that shall be defined to have the following meanings:

It must be noted that, as used in the specification and the appended claims, the singular forms “a”, “an” and “the” include plural references unless the context clearly dictates otherwise. Thus, for example, reference to “a surfactant” includes mixtures of two or more such surfactants, and the like.

As used herein, the term “about” indicates that a value can vary by up to +5%, +2%, or ±1.

As used herein, the term “body” means the physical whole structure of a person or an animal.

As used herein, “percent solids” has its standard meaning of the quantification of the non-volatile ingredients in a composition.

As used herein, “aqueous media” refers to a spectrum of water-based solutions including, but not limited to, homogeneous solutions in water with solubilized components, cell media solutions, buffer solutions, isotonic solutions, salt solutions, emulsified solutions, surfactant solutions, amniotic fluids, Wharton's jelly, serum, hydrophilic polymer solutions, and viscous or gelled homogeneous or emulsified solutions in water.

As used herein, “surfactant” has its standard meaning and includes compounds that lower the surface tension (or interfacial tension) between two liquids or between a liquid and a solid and includes emulsifying agents, emulsifiers, detergents, wetting agents, and surface-active agents.

As used herein, “biologically active agents” has its standard meaning and includes chemical or biological substances or formulations that beneficially affect human or animal health and well-being or are intended for use in the cure, mitigation, treatment, prevention, or diagnosis of infection or disease, or are destructive to or inhibit the growth of microorganisms.

In some aspects, a hydrolyzed collagen-based composition is provided that includes hydrolyzed collagen and 0.0005 wt % to 50 wt % of antibiotic component based on the percent solids of the hydrolyzed collagen-based composition.

In some embodiments, the hydrolyzed collagen-based composition comprises 25 to 99.9995 wt % of hydrolyzed collagen. In some embodiments, the hydrolyzed collagen-based composition comprises up to 90 wt % or up to 99 wt % of hydrolyzed collagen based on the percentage of solids in the composition. In some embodiments, the hydrolyzed collagen-based composition comprises at least 50 wt %, at least 60 wt %, at least 70 wt %, at least 80 wt %, or at least 90 wt % of hydrolyzed collagen based on the percentage of solids in the hydrolyzed collagen-based composition.

In some embodiments, the hydrolyzed collagen-based compositions can include an antibiotic component that includes polyene antimycotics, aminoglycosides, glycopeptide antibiotics, beta-lactam antibiotics, or combinations thereof.

In some embodiments, the polyene antimycotics can be selected from amphotericin B, nystatin and natamycin, and combinations thereof.

In some embodiments, the aminoglycoside antibiotics can be selected from gentamicin, amikacin, neomycin, kanamycin A, plazomicin, netilmicin, tobramycin, paromomycin, streptomycin, and combinations thereof.

In some embodiments, the beta-lactam antibiotics can be selected from cephalosporin, methicillin, amoxicillin, carbapenems, and combinations thereof.

In some embodiments, the glycopeptide antibiotics can be selected from vancomycin, oritavancin, dalbavancin, telavancin, and combinations thereof.

In some embodiments, the source of collagen used to form the hydrolyzed collagen can be human, bovine, porcine, piscine, ovine, avian, another source, or combinations thereof. In some embodiments, the hydrolyzed collagen is derived from bovine collagen.

In some embodiments, the hydrolyzed collagen-based composition comprises 0.0005 wt % to 50 wt-% of antibiotics based on the percent solids of the composition. In some embodiments, the hydrolyzed collagen-based composition comprises 0.0005 to 25 wt-% of antibiotics based on the percent solids of the composition. In some embodiments, the hydrolyzed collagen-based composition comprises an antibiotic component in an amount of at least 0.005 wt-%, at least 0.01 wt-%, at least 0.05 wt-%, at least 0.5 wt-%, at least 5 wt-%, or at least 25 wt-%, based on the percent solids of the hydrolyzed collagen-based composition. In some embodiments, the hydrolyzed collagen-based composition comprises an antibiotic component in an amount of up to 50 wt-%, up to 40 wt-%, up to 30 wt-%, up to 25 wt-%, or up to 20 wt-%, or up to 15 wt-%, or up to 10 wt-%.

Antibiotics useful in the hydrolyzed collagen-based compositions include the categories: bacteriostatic, bactericidal, and antimycotics. Bacteriostatic antibiotics can be further classified into glycylcyclines, tetracylines, lincosamides, macrolides, oxazolidinones, and sulfonamides while bactericidal antibiotics include aminoglycosides, beta-lactams, fluoroquinolines, glycopeptides, and nitroimidazoles.

Polyene antimycotics, aminoglycoside antibiotics, glycopeptide antibiotics, and their derivatives and combinations thereof are antibiotics that are effective against a broad range of microbes which include fungi, yeast, Gram-negative and Gram-positive bacteria, protozoa, and viruses.

In some embodiments, the antibiotic component comprises a polyene antimycotic. In some embodiments, the antibiotic component includes a polyene antimycotic selected from amphotericin B, nystatin, natamycin, a derivative thereof, or a combination thereof. In some embodiments, the antibiotic component includes amphotericin B or a derivative thereof. In some embodiments, polyene antimycotics are present in an amount of at least 0.005 wt-%, at least 0.05 wt-%, at least 0.5 wt-%, at least 5 wt-%, at least 15 wt-%, or at least 25 wt-%, based on the percent solids of the composition.

Polyene antimycotics are usually derived from species of Streptomyces bacteria and function by attaching to the ergosterol component of fungal cell membranes, hence causing fungal death. Amphotericin B is an example of the polyene class of antifungals.

Amphotericin B, a cyclic amphoteric molecule, is an effective anti-fungal and anti-protozoan agent with a low incidence of drug resistance. Amphotericin B is biosynthetically produced but was originally isolated from a Venezuelan soil bacterium, Streptomyces nodosus. Its principal mechanism of action is to infiltrate fungal, and mammalian, cell membranes at sterol-containing sites to form pores and, hence, damaging at least cell membrane barrier function. Fungal cell membranes contain ergosterol which is more reactive with Amphotericin B than mammalian cell membranes which contain cholesterol and, thus, when delivered systemically or locally fungi are preferentially affected (Vertut-Croquin 1983). Amphotericin B may be complexed with sodium deoxycholate, cholesteryl sulfate, lipids, liposomes, and combinations thereof.

In some embodiments, the antibiotic component comprises an aminoglycoside antibiotic. In some embodiments, the aminoglycoside antibiotic is selected from gentamicin, amikacin, neomycin, kanamycin A, plazomicin, netilmicin, tobramycin, paromomycin, streptomycin, derivatives thereof, or a combination thereof. In some embodiments, the hydrolyzed collagen-based composition comprises gentamicin or a derivative thereof. In some embodiments, the hydrolyzed collagen-based composition comprises an aminoglycoside antibiotic in an amount of at least 0.005 wt-%, at least 0.05 wt-%, at least 0.5 wt-%, at least 5 wt-%, at least 15 wt-%, or at least 25 wt-%, based on the percent solids of the composition.

Aminoglycoside antibiotics are effective antibiotics against a relatively broad spectrum of aerobic micro-organisms, including Gram-negative bacteria and mycobacteria. The mechanism of action is postulated to be attachment to bacterial ribosomal 306 subunit, hence, leading to misreading of the genetic code, and protein synthesis is disrupted.

Gentamicin, representative of aminoglycoside antibiotics, is commonly used to systemically treat bacterial bone infections, meningitis, pneumonia, and sepsis, for instance. Topically it is used to treat various wounds and burns. Gentamicin, an aminoglycoside, is primarily effective against Gram-negative bacteria but is also an effective antibiotic for Gram-positive Staphylococcus. Gentamicin, an aminoglycoside with multiple pendant amino groups and hydroxyl groups functions by disrupting bacterial protein synthesis creating mistranslated proteins, slowing protein elongation rate (Aguirre 2021), and deactivating ribosomes (Borovinskaya 2007). It is synthesized via fermentation of Micromonospora purpurea and, hence, is a complex mixture of biochemicals. For the purposes of this invention, Gentamicin encompasses the entire mixture as well as individual components used singly or together. Gentamicin can be injected intravenously or intramuscularly as well as topically applied as a liquid, gel, paste, foam, spray, incorporated into dressing materials or coated onto medical devices.

In some embodiments, the antibiotic component comprises a glycopeptide antibiotic. In some embodiments, the glycopeptide antibiotic is selected from vancomycin, oritavancin, dalbavancin, telavancin, derivatives thereof, and combinations thereof. In some embodiments, the hydrolyzed collagen-based composition comprises vancomycin or a derivative thereof. In some embodiments, the hydrolyzed collagen-based composition comprises a glycopeptide antibiotic in an amount of at least 0.005 wt-%, at least 0.05 wt-%, at least 0.5 wt-%, at least 5 wt-%, at least 15 wt-%, or at least 25 wt-%, based on the percent solids of the composition.

Glycopeptide antibiotics are effective antibiotics, which are glycosylated cyclic or polycyclic nonribosomal peptides. Glycopeptide antibiotics inhibit aerobic and anaerobic Gram-positive bacterial peptidoglycan synthesis. More recent glycopeptides have been expanded to include lipoglycopeptides which utilize the lipid component to penetrate cell membranes.

Vancomycin, one of the original glycopeptide antibiotics, is reserved as a treatment for life-threatening Gram-positive bacterial infections (MRSA, MRSE, C. difficile for instance) that are unresponsive to other antibiotics. Vancomycin functions by binding to the peptide motif, D-Ala-D-Ala, which then disrupts Gram-positive cell wall synthesis and, hence, cell death occurs (Mühlberg 2020). Vancomycin was also first found in a soil bacteria, but from Borneo. Intravenous injections are preferred but topical, oral and rectal routes may be used depending on the infection environment and patient needs. For the purposes of this invention, Vancomycin encompasses the natural product as well as variants and derivatives, such as modification with cationic oligopeptides.

Beta-lactam antibiotics are effective against Gram-negative and Gram-positive bacteria. Bacterial cell walls comprise peptidoglycans as a structural component for, at least, mechanical stability. Beta-lactams inhibit synthesis of these peptidoglycans through prevention of peptide crosslinking to form peptidoglycans. Thus, bacterial cell wall stability is decreased which results in lysis and loss of bacterial cell viability.

In some embodiments, the antibiotic component comprises a beta-lactam antibiotic. In some embodiments, the antibiotic component includes a beta-lactam antibiotic selected from cephalosporins, penicillins, carbapenems, monobactams, and beta-lactamase inhibitors, derivatives thereof and combinations thereof. In some embodiments, the antibiotic component includes cephalosporin or a derivative thereof. In some embodiments, beta-lactam antibiotics are present in an amount of at least 0.003 wt %, at least 0.05 wt %, at least 5 wt %, at least 15 wt %, or at least 25 wt % based on the percent solids of the composition.

Cephalosporins are frequently delivered via injection, orally or topically.

In some embodiments, the antibiotic comprises Amphotericin B and its derivatives.

In some embodiments, the antibiotic comprises Gentamicin as a complex mixture or parts thereof.

In some embodiments, the antibiotic comprises Vancomycin and its variants and derivatives.

In some embodiments, the antibiotic comprises Cephalosporin and its derivatives.

In some embodiments, the antibiotic comprises combinations of Amphotericin B, Gentamicin, Vancomycin, and Cephalosporin.

In some embodiments, the antibiotic component comprises at least two antibiotics independently selected from polyene antimycotics, aminoglycoside antibiotics, beta-lactam antibiotics, and glycopeptide antibiotics. In some embodiments, the antibiotic component comprises at least two selected from Amphotericin B or derivatives thereof, Gentamicin or derivatives thereof, Vancomycin or derivatives thereof, or Cephalosporin or derivatives thereof.

In some embodiments, the antibiotic component comprises at least three antibiotics independently selected from polyene antimycotics, aminoglycoside antibiotics, beta-lactam antibiotics and glycopeptide antibiotics. In some embodiments, the antibiotic component comprises at least three selected from Amphotericin B or derivatives thereof, Gentamicin or derivatives thereof, Vancomycin or derivatives thereof, or Cephalosporin or derivatives thereof.

In some embodiments, the antibiotic component comprises at least four antibiotics, each of which is independently selected from polyene antimycotics, aminoglycoside antibiotics, beta-lactam antibiotics and glycopeptide antibiotics. In some embodiments, the antibiotic component comprises at least four antibiotics, each of which is independently selected from Amphotericin B or derivatives thereof, Gentamicin or derivatives thereof, Vancomycin or derivatives thereof, or Cephalosporin or derivatives thereof.

In some embodiments, the antibiotic component comprises at least one antibiotic from each of the following classes of antibiotics: polyene antimycotics, aminoglycoside antibiotics, beta-lactam antibiotics and glycopeptide antibiotics. In some embodiments, the antibiotic component comprises Amphotericin B or derivatives thereof, Gentamicin or derivatives thereof, Vancomycin or derivatives thereof, and Cephalosporin or derivatives thereof.

In some embodiments, a ratio between any two components of the antibiotic component that are present is in a range from 1:200 to 200:1, based on weight of the components. In some embodiments, the weight ratio of any two components of the antibiotics is from 1:100 to 100:1, from 1:50 to 50:1, from 1:20 to 20:1, or any combination of these.

In some embodiments, the weight ratio of Amphotericin B to Gentamicin to Vancomycin to Cephalosporin ranges from 200 to 0 parts of each antibiotic. In some embodiments, the ratio of Amphotericin B to Gentamicin to Vancomycin to Cephalosporin ranges from 40 to 0 parts of each antibiotic, or 25 to 0 parts of each antibiotic, or from 2 to 0 parts of each antibiotic. In some embodiments, the ratio of Amphotericin B to Gentamicin to Vancomycin to Cephalosporin ranges from 40 to 1 parts of each antibiotic, or 25 to 1 parts of each antibiotic, or from 2 to 0.25 parts of each antibiotic.

In some embodiments, the composition comprises Amphotericin B to Gentamicin to Vancomycin to Cephalosporin in a ratio from 1:1:1:1 to 1:50:50:50, or from 1:1:1:1 to 50:1:50:50, or from 1:1:1:1 to 50:50:1:50, or from 1:1:1:1 to 50:50:50:1, or from 1:1:1:1 to 1:1:50:50, or from 1:1:1:1 to 50:1:50:1, or from 1:1:1:1 to 50:50:1:1, or from 1:1:1:1 to 1:50:1:50, or from 1:1:1:1 to 1:1:1:50, or from 1:1:1:1 to 1:50:1:1, or from 1:1:1:1 to 1:1:50:1, or from 1:1:1:1 to 50:1:1:1. In some embodiments, the ratio of any two of these components is as described above (e.g., from 1:200 to 200:1, from 1:100 to 100:1, from 1:50 to 50:1, from 1:20 to 20:1, or any combination of these). In some embodiments, the ratio of any two or three of Amphotericin B to Gentamicin to Vancomycin to Cephalosporin can be in a range from those selected above to the exclusion of the remaining two or one, respectively, of Amphotericin B to Gentamicin to Vancomycin to Cephalosporin. For example, the ratio of Amphotericin B to Vancomycin to Cephalosporin can range from 1:1:1 to 1:50:50, or from 1:1:1 to 50:50:1, or from 1:1:1 to 50:1:50, or from 1:1:1 to 1:1:50, or from 1:1:1 to 1:1:50, or from 1:1:1 to 1:1:1, or from 1:1:1 to 1:50:1, or from 1:1:1 to 50:1:1.

Similar adjustments can be made for any other combination of two or three of Amphotericin B to Gentamicin to Vancomycin to Cephalosporin. Examples of ratios of two antibiotics can include Amphotericin B to Gentamicin, Amphotericin B to Vancomycin, Amphotericin B to Cephalosporin, Gentamicin to Vancomycin, Gentamicin to Cephalosporin, and Vancomycin to Cephalosporin.

Examples of ratios of three antibiotics can include Amphotericin B to Gentamicin to Vancomycin, Amphotericin B to Gentamicin to Cephalosporin, Amphotericin B to Vancomycin to Cephalosporin, and Gentamicin to Vancomycin to Cephalosporin.

In some embodiments, the antibiotic component comprises more Amphotericin B than Gentamicin or Vancomycin or Cephalosporin.

In some embodiments, the antibiotic component comprises more Gentamicin than Amphotericin B or Vancomycin or Cephalosporin.

In some embodiments, the antibiotic component comprises more Vancomycin than Amphotericin B or Gentamicin or Cephalosporin.

In some embodiments, the antibiotic component comprises more Cephalosporin than Amphotericin B or Gentamicin or Vancomycin.

Although the foregoing refers to amphotericin B, gentamicin, vancomycin, and cephalosporin, the compositions and ratios are equally applicable for the corresponding classes of antibiotics. For instance, any ratio described herein between two or more of Amphotericin B to Gentamicin to Vancomycin to Cephalosporin can apply equally to any ratio based on a polyene antimycotic to an aminoglycoside to a glycopeptide to a beta-lactam.

In some embodiments, the hydrolyzed collagen-based composition exhibits a kill of at least 1 log reduction of a planktonic microbe. In some embodiments, the composition exhibits a kill of at least 2 log reduction, or at least 3 log reduction, or at least 4 log reduction, or at least 5 log reduction, or at least 6 log reduction of a planktonic microbe. In some embodiments, the planktonic microbe is at least one of a gram-negative bacteria, a gram-positive bacteria, a yeast, a fungi, a protozoan, or a virus. In some embodiments, the planktonic microbe is at least one of P. aeruginosa, C. albicans, or methicillin-resistant S. aureus (MRSA). In some embodiments, the planktonic microbe is a gram-negative bacteria, such as P. aeruginosa. In some embodiments, the planktonic microbe is a yeast, such as C. albicans. In some embodiments, the planktonic microbe is gram-positive bacteria, such as methicillin-resistant S. aureus (MRSA).

In some embodiments, the hydrolyzed collagen-based composition enhances a natural tissue regeneration process. In some embodiments, the natural tissue regeneration process that is enhanced is at least one of cell migration, cell proliferation, cell viability, cell differentiation, and angiogenesis. In some embodiments, the cells are fibroblasts. As used herein, a natural tissue regeneration process is enhanced if the property is improved compared to an untreated control. In some embodiments, at least one natural tissue regeneration process is improved by at least 2%, or at least 5%, or at least 10% compared to an untreated control.

In some embodiments, the hydrolyzed collagen-based composition comprises at least one additional ingredient selected from glycolipids, glycoproteins, immunological response modifiers, saccharides, steroids, and polysaccharides.

In some embodiments, the hydrolyzed collagen-based composition has a form selected from a powder, a liquid, a gel, a paste, a cream, a suspension, an emulsion, a film, a sheet, a foam, a lotion, a spray, an aerosol, a capsule, or a tablet. In some embodiments, the composition is a powder.

In some embodiments, the antibiotic component may be incorporated in the hydrolyzed collagen-based composition using a variety of methods such as blending the antibiotic as a powder or as a solution, using a solvent such as aqueous media. If a solution is used, the solvent can be evaporated or can remain in the final form.

In some embodiments, antibiotic powder can simply be mixed with hydrolyzed collagen powder. The powder can then be applied directly. Alternately, the powder can be dissolved (e.g., within a syringe) in an aqueous solvent, then applied as appropriate.

In some embodiments, antibiotic powder can be mixed with hydrolyzed collagen powder, then dissolved in an aqueous solvent, and then dried and formulated into a combined powder.

In some embodiments, antibiotic powder can be mixed with hydrolyzed collagen powder and any other solid ingredients to form the hydrolyzed collagen-based composition.

In some embodiments, the hydrolyzed collagen-based composition, including the antibiotic component, provides preservative efficacy for the formulation before use. During treatment, the hydrolyzed collagen-based composition imparts preventative protection from infection as well as antibiotic activity to treat or prevent a microbial infection.

In some embodiments, the biocompatibility of the hydrolyzed collagen-based compositions described herein lends itself to assisting with cellular and tissue healing, as well as, protection from or treatment of infection.

In some embodiments, any of the hydrolyzed collagen-based compositions described herein can be mixed with polar liquids, such as alcohols and water, and applied to or within living tissue; or in, on, or surrounding a device (e.g., an implant device, a tissue replacement product, etc.).

In some embodiments, the antimicrobial hydrolyzed collagen-based compositions described herein can be aqueous compositions. As used herein, “aqueous” compositions include, but are not limited to, solutions in water with solubilized components, emulsified solutions in water stabilized by surfactants or hydrophilic polymers, as well as, viscous or gelled homogeneous or emulsified solutions.

In some embodiments, the hydrolyzed collagen-based composition comprises a fluid selected from the group consisting of water, isotonic saline, balanced salt solution, buffer solution, Ringer's solution, cell culture media, stem cell media, serum, plasma, amniotic fluid, Wharton's jelly, nutrient broth, antiseptic solutions, and combinations thereof.

In some embodiments, the hydrolyzed collagen-based composition can be utilized in a powder state and can be placed into or on a tissue defect, wound, burn, or in, on, or surrounding a medical device (e.g., an implanted medical device or medical device prior to implantation). In some embodiments, the powder mixture can be hydrated by endogenous or exogenous fluid sources.

Examples of tissue defects (e.g., impaired tissue) that can be treated by the hydrolyzed collagen-based compositions described herein include, but are not limited to, degraded tissue, surgical incision sites, lesions, fissures, fistulas and diverticula. These tissue defects can be physiological or the result of infection, inflammation, surgery, cyst, tumor removal, or traumatic injury or remodeling of soft tissue or hard tissue, such as in skin and wound healing, plastic surgery, cosmetic surgery, reconstructive surgery, coating/sealing of tissue replacement products, tendon repair, hernia repair, craniofacial surgery, ophthalmic surgery, cervicofacial rhytidectomy, abdominoplasty, breast augmentation, myocardium repair, cartilage repair, bone repair, joint repair, nerve repair, spinal cord repair, liver tissue regeneration, bladder repair, muscle repair, mastopexy, rheumatology, gynecomastia reduction, body contouring, skin rejuvenation, skin resurfacing, microsurgery, dermato-cosmetics for filling in wrinkles, masking scars or enhancing lips, and the like.

Examples of implanted medical devices referenced herein include, but are not limited to joint implants, bone repair implants (e.g., rods, screws, etc.), tissue implants, tissue replacement products, heart and cardiovascular devices, pain control devices, neurological control devices, dental implants, and the like.

When the antimicrobial hydrolyzed collagen-based composition is applied to a biological substrate or medical device in either a hydrated or dry form, the mixture (based on weight percent solids) can contain the antibiotic component at a concentration of 0.0005 wt % to 50 wt %, or from 0.005 wt % to 25 wt %, from 0.05 wt % to 5 wt %, from 0.05 wt % to 1 wt %. The mixture (based on weight percent solids) can contain hydrolyzed collagen at a concentration of 25 wt % to 99.9995 wt %, from 50 wt % to 99.99 wt %, from 80 wt % to 99.9 wt %, from 95 wt % to 99.9 wt %, or from 99 wt % to 99.9 wt %.

In some embodiments, the hydrolyzed collagen-based composition can include a hydrophobic vicinal diol as a further antimicrobial component. In some embodiments, the hydrophobic vicinal diol can be a monoalkyl glycol, a glycerol alkyl ether, a monoacyl glycerol, or a combination thereof. In some embodiments, the hydrophobic vicinal diol is present at a concentration of from 0.05 wt % to 20 wt %, or from 0.5 wt % to 18 wt %, or from 1 wt % to 15 wt % based on weight percent solids.

In some embodiments, the hydrolyzed collagen-based composition comprises at least one hydrophobic monoalkyl glycol, a hydrophobic glycerol alkyl ether, and a hydrophobic monoacyl glycerol. In addition to being branched or unbranched, these compounds can either be saturated or unsaturated.

In some embodiments, the monoalkyl glycol comprises at least one of caprylyl glycol (also known as SC-10®, 1,2-dihydroxyoctane, 1,2-octanediol, and 1,2-octylene glycol), hexylene glycol, 2-methyl-2,4-pentanediol, 1,3-butylene glycol, triethylene glycol, glycol bis(hydroxyethyl) ether. In some embodiments, the monoalkyl glycols include caprylyl glycol, a component of Sensiva® SC 10. In some embodiments, the monoalkyl glycols include glycerol 1-(2-ethylhexyl) ether (2-ethylhexylglycerin).

Sensiva® SC 10 is reported to combine the excellent skin care and deodorizing properties of 2-ethylhexylglycerin (Sensiva® SC 50) with the moisturizing and antimicrobial properties of caprylyl glycol. Additionally, Sensiva® SC 10 can contribute to the antimicrobial stability of cosmetic formulations. It can also be used to improve the efficacy of traditional cosmetic preservatives, such as parabens or phenoxyethanol (Schulke & Mayr, Sensiva® SC 10 Multifunctional Cosmetic Ingredient). Screening tests with Sensiva® SC 10 have shown that it reliably inhibits the growth and multiplication of Gram-positive odor causing bacteria, while at the same time it does not affect beneficial skin flora.

In some embodiments, Sensiva® SC 10 (caprylyl glycol) by Schulke & Mayr may be present in an amount of 0.05 to 5 wt-% based on solids in the composition. In some embodiments, Sensiva® SC 10 may be present in an amount of 0.1 to 4 wt-% based on solids in the composition.

In some embodiments, the hydrophobic glycerol alkyl ether includes at least one of 1-O-heptylglycerol, 1-O-octylglycerol, 1-O-nonylglycerol, 1-O-decylglycerol, 1-O-undecylglycerol, 1-O-dodecylglycerol, 1-O-tridecylglycerol, 1-O-tetradecylglycerol, 1-O-pentadecylglycerol, 1-O-hexadecylglycerol (chimyl alcohol), 1-O-heptadecylglycerol, 1-O-octadecylglycerol (batyl alcohol), 1-O-octadec-9-enyl glycerol (selachyl alcohol), glycerol 1-(2-ethylhexyl) ether (also known as octoxyglycerin, 2-ethylhexyl glycerin, 3-(2-ethylhexyloxy)propane-1,2-diol, and Sensiva® SC 50 (2-ethylhexylglycerin), 2-ethylhexyl diglycol ether, 2-ethylhexyl oligoglycol ethers, glycerol 1-heptyl ether, glycerol 1-octyl ether, glycerol 1-decyl ether, glycerol 1-dodecyl ether, glycerol 1-tridecyl ether, glycerol 1-tetradecyl ether, glycerol 1-pentadecyl ether, glycerol 1-hexadecyl ether, or glycerol 1-octadecyl ether. In some embodiments, the hydrophobic glycerol alkyl ether is selected from glycerol 1-(2-ethylhexyl) ether, (Sensiva® SC 50) and 1-O-dodecylglycerol. In some embodiments, the hydrophobic glycerol alkyl ether is glycerol 1-(2-ethylhexyl) ether.

Sensiva® SC 50 reliably inhibits the Gram-positive odor-causing bacteria on the skin and is used in deodorant formulations. It is reported to boost the efficacy of traditional preservatives. In some embodiments, Sensiva® SC 50 (2-ethylhexylglycerin) by Schulke & Mayr may be present in an amount of 0.5 to 15 wt-% based on solids in the composition. In some embodiments, Sensiva® SC 50 may be present in an amount of 0.3 to 12 wt-% based on solids in the composition.

In some embodiments, the hydrophobic monoacyl glycerol includes at least one of 1-O-decanoylglycerol (monocaprin), 1-O-undecanoylglycerol, 1-O-undecenoylglycerol, 1-O-dodecanoylglycerol (monolaurin, also called glycerol monolaurate and Lauricidin®), 1-O-tridecanoylglycerol, 1-O-tetradecanoylglycerol (monomyristin), 1-O-pentadecanoylglycerol, 1-O-hexadecanoylglycerol, 1-O-heptadecanoylglycerol, and 1-O-octanoylglycerol (monocaprylin). In some embodiments, the hydrophobic monoacyl glycerol is selected from 1-O-decanoylglycerol, 1-O-dodecanoylglycerol, 1-O-tetradecanoylglycerol, and 1-O-octanoylglycerol. In some embodiments, the hydrophobic monoacyl glycerol is 1-O-dodecanoylglycerol. In some embodiments, glycerols substituted in the 1-O-position can be preferred over those substituted in the 2-O-position, or disubstituted in both the 1-0 and 2-0 positions.

Since the antimicrobial hydrophobic monoalkyl vicinal diol and an antimicrobial hydrophobic monoalkyl and monoacyl glycerol have hydrophilic —OH groups but low or negligible water solubility, in some embodiments a surfactant can be added to aid in solution compatibilization and homogeneity of these compounds.

In some embodiments, the hydrolyzed collagen-based composition includes at least one anti-infective agent. In some embodiments, anti-infective agents include, but are not limited to, biguanides, such as poly(hexamethylene biguanide) (PHMB) and its salts, chlorhexidine and its salts, such as chlorhexidine digluconate, and alexidine and its salts, such as alexidine dihydrochloride, benzalkonium chloride, benzethonium chloride, cetyltrimethylammonium bromide, capryl glycol. In some embodiments, the anti-infective agent comprises at least one of PHMB or capryl glycol.

The hydrolyzed collagen-based composition can include a metal chelating agent, a surfactant, or both. Where the hydrolyzed collagen-based composition is an aqueous solution, a water-soluble polymer can be added to increase solution viscosity, to change rheology, and/or to prolong residence time of the antimicrobial composition on a biological surface or medical device.

In some embodiments, the hydrolyzed collagen-based composition comprises a water-soluble polymer. In some embodiments, the water-soluble polymers are present at a concentration of from 0.01 weight % to 60 weight %, based on the percent solids of the hydrolyzed collagen-based composition. In some embodiments, the hydrolyzed collagen-based compositions include water-soluble polymers in amounts ranging from about 0.1 to about 55 wt %, from 0.25 to 50 wt %, from 0.5 to 25 wt %, or from 1.0 to 10.0 wt %, based on the percent solids of the hydrolyzed collagen-based composition.

In some embodiments, the water-soluble polymers are selected from the group consisting of poly(ethylene glycol), poly(ethylene oxide), poly(vinyl alcohol) and copolymers, poly(N-vinylpyrrolidone) and copolymers, methylcellulose, hydroxyethylcellulose, hydroxypropylcellulose, hydroxypropylmethylcellulose, carboxymethylcellulose, guar gum, hydroxyethylguar, hydroxypropylguar, gelatin, albumin, hydroxypropylmethylguar, carboxymethylguar, carboxymethylchitosan, locust bean gum, carrageenan, xanthan gum, gellan gum, pullulan, alginate, chondroitin sulfate, maltodextran, dextran, dextran sulfate, Aloe vera gel, scleroglucan, schizophyllan, gum arabic, tamarind gum, poly(methyl vinyl ether), ethylene oxide-propylene oxide-ethylene oxide block copolymers, hyaluronan, chondroitin sulfate, keratan sulfate, dermatan sulfate, heparan sulfate, dextran, carbomer and its salts, poly(acrylic acid) and its salts, poly(methacrylic acid) and its salts, poly(ethylene-co-acrylic acid), poly(vinyl methyl ether), poly(vinylphosphoric acid) salts, poly(vinylsulfonic acid) salts, sodium poly(2-acrylamido-2-methylpropanesulfonate), polyacrylamide(s), poly(N,N-dimethylacrylamide), poly(N-vinylacetamide), poly(N-vinylformamide), poly(2-hydroxyethyl methacrylate), poly (glyceryl methacrylate), poly(2-ethyl-2-oxazoline), poly(N-isopropylacrylamide) and poly(N-vinylcaprolactam), the latter two hydrated below their Lower Critical Solution Temperatures, polyquaternium-1, polyquaternium-6, poly-quaternium-10, ionene polymers, cationic guar, pyridinium polymers, imidazolium polymers, diallyldimethylammonium polymers, acryloyl-, methacryloyl-, and styryl-trimethylammonium polymers, acrylamido- and methacrylamido-trimethylammonium polymers, and derivatives and combinations thereof.

In some embodiments, the hydrolyzed collagen-based composition can include a chelating agent. Chelating agents can enhance the susceptibility of bacteria and other organisms to the biocidal effects of the antibiotic. Thus, a hydrolyzed collagen-based composition containing a chelating agent can be more effective in combating infection than one without a chelating agent. Additionally, chelating agents deactivate matrix metalloproteases (MMPs), enzymes that can impede tissue formation and healing by breaking down collagen. MMPs are often found at elevated levels in impaired tissue. Chelating agents bind to zinc ions, which are necessary for MMP activity, disrupting the MMP, causing deactivation, and thus facilitating healing.

In some embodiments, the chelating agent is selected from any compound that is suitable for medical or veterinary use and is able to sequester monovalent or polyvalent metal ions. Metal ion examples include, but are not limited to, sodium, lithium, rubidium, cesium, calcium, magnesium, barium, cerium, cobalt, copper, iron, manganese, nickel, strontium or zinc. The outermost surface of bacterial cells universally carries a net negative charge, which is usually stabilized by divalent cations such as Mg²⁺ and Ca²⁺. This is associated with the teichoic acid and polysaccharide elements of Gram-positive bacteria, the lipopolysaccharide of Gram-negative bacteria, and the cytoplasmic membrane itself. Thus, the chelating agent aids in destabilizing microorganisms. Additionally, the chelating agent may deactivate matrix metalloproteases, such as in inflammatory wounds, facilitating collagen development.

Suitable chelating agents comprise, but are not limited to, aminocarboxylic acids, citric acid, ethylenediaminetetraacetic acid (EDTA), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N′-bis(2-hydroxybenzyl)ethylenediamine-N,N′-diacetic acid, ethylenediamine-N,N′-diacetic acid, ethylenediamine-N,N′-dipropionic acid, triethylenetetraaminehexaacetic acid, ethylenediamine-N,N′-bis(methylenephosphonic acid), iminodiacetic acid, N,N′-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid and biphosphonates such as editronate, and salts thereof. Suitable chelating agents include, for example but are not limited to, hydroxyalkylphosphonates as disclosed in U.S. Pat. No. 5,858,937, specifically the tetrasodium salt of 1-hydroxyethylidene-1,1-diphosphonic acid, also referred to as tetrasodium etidronate, commercially available from Monsanto Company as DeQuest 2016 diphosphonic acid sodium salt or phosphonate.

Especially preferred chelating agents are mixed salts of EDTA such as disodium, trisodium, tetrasodium, dipotassium, tripotassium, tetrapotasssium, lithium, dilithium, ammonium, diammonium, triammonium, tetraammonium, calcium and calcium-disodium. In some embodiments, the chelating agent can be or include a disodium, trisodium or tetrasodium salt of EDTA. In some embodiments, the chelating agent can be or can include disodium EDTA and/or trisodium EDTA.

In some embodiments, the hydrolyzed collagen-based composition may comprise 0.01 wt % to 5 wt % of a chelating agent or 0.05 wt %-3 wt % based on the percent solids of the composition.

Suitable surfactants include, but are not limited to, cationic, anionic, nonionic, amphoteric and ampholytic surfactants. Preferred surfactants are nonionic and amphoteric surfactants. The surfactants can have an HLB (hydrophilic-lipophilic balance) value of 18-30 in order to maintain the biocidal activity of the antibiotics, while facilitating a non-cytotoxic composition. The surfactant lowers surface tension, facilitating wetting of a surface for enhanced activity of the biocidal agent and for assistance with debridement.

Suitable nonionic surfactants include ethylene oxide/propylene oxide block copolymers of poloxamers, reverse poloxamers, poloxamines, and reverse poloxamines. In some embodiments, poloxamers and poloxamines are preferred, with poloxamers generally being most preferred. Poloxamers and poloxamines are available from BASF Corp. under the trade names of Pluronic® and Tetronic®.

Suitable Pluronic surfactants include but are not limited to Pluronic F38 having a HLB of 31 and average molecular weight (AMW) of 4,700, Pluronic F68 having a HLB of 29 and AMW of 8,400, Pluronic 68LF having a HLB of 26 and AMW or 7,700, Pluronic F77 having a HLB of 25 and AMW of 6,600, Pluronic F87 having a HLB of 24 and AMW of 7,700, Pluronic F88 having a HLB of 28 and AMW or 11,400, Pluronic F98 having a HLB of 28 and AMW of 13,000, Pluronic F108 having a HLB of 27 and AMW of 14,600, Pluronic F127 (also known as Poloxamer 407) having a HLB of 18-23 and AMW of 12,600, and Pluronic L35 having a HLB of 19 and AMW of 1,900.

Another class of surfactant is that of the diamino block copolymers of ethylene oxide and propylene oxide sold under the trade name Tetronic®. An exemplary surfactant of this type is Tetronic 1107 (also known as Poloxamine 1107).

In addition to the above, other surfactants may be added, such as for example polyethylene glycol esters of fatty acids, e.g., coconut, polysorbate, polyoxyethylene or polyoxypropylene ethers of higher alkanes (C12-C18), polysorbate 20 available under the trademark Tween 20, polyoxyethylene (23) lauryl ether available under the trademark Brij 35, polyoxyethylene (40) stearate available under the trademark Myrj 52, and polyoxyethylene (25) propylene glycol stearate available under the trademark Atlas G 2612. Other neutral surfactants include nonylphenol ethoxylates such as nonylphenol ethoxylates, Triton X-100, Brij surfactants of polyoxyethylene vegetable-based fatty ethers, Tween 80, decyl glucoside, and lauryl glucoside.

Amphoteric surfactants suitable for use in the hydrolyzed collagen-based compositions described herein can include materials of the type offered commercially under the trademark Miranol. Another useful class of amphoteric surfactants is exemplified by cocoamidopropyl betaine, commercially available from various sources.

Emollients/moisturizers and humectants can be added to the hydrolyzed collagen-based compositions to provide a more soothing antibiotic composition when used topically. Emollients/moisturizers function by forming an oily layer on the top of the skin that traps water in the skin. Petrolatum, lanolin, mineral oil, dimethicone, and siloxy compounds are common emollients. Other emollients include isopropyl palmitate, isopropyl myristate, isopropyl isostearate, isostearyl isostearate, diisopropyl sebacate, propylene dipelargonate, 2-ethylhexyl isononoate, 2-ethylhexyl stearate, cetyl lactate, lauryl lactate, isopropyl lanolate, 2-ethylhexyl salicylate, cetyl myristate, oleyl myristate, oleyl stearate, oleyl oleate, hexyl laurate, and isohexyl laurate, lanolin, olive oil, cocoa butter, shea butter, octyldodecanol, hexyldecanol, dicaprylyl ether and decyl oleate.

Humectants include glycerin, lecithin, 1,2-propylene glycol, dipropylene glycol, polyethylene glycol, 1,3-butylene glycol, and 1,2,6-hexanetriol. Humectants function by drawing water into the outer layer of skin.

Anti-inflammatory agents can also be added, such as water-soluble derivatives of aspirin, vitamin C, methylsulfonylmethane, tea tree oil, and non-steroidal anti-inflammatory drugs.

It is often desirable to include water-soluble viscosity builders in the hydrolyzed collagen-based compositions described herein, particularly when the hydrolyzed collagen-based composition includes (or will hydrate to include) an aqueous phase. Because of their demulcent effect and possible hydrophobic interactions with biological tissue, water-soluble polymers have a tendency to enhance the interaction with a biological tissue by means of a hydrated film on the surface. Because of this behavior, such water-soluble polymers can increase the residence time of the hydrolyzed collagen composition or gel on a biological tissue. Aqueous media may be incorporated into the hydrolyzed collagen-based composition or may be derived from a treatment surface that is wet with, for example, wound exudate, blood, or plasma.

Water-soluble viscosity builders useful herein include, but are not limited to, methylcellulose, hydroxyethylcellulose, hydroxypropylcellulose, hydroxypropylmethylcellulose, carboxymethylcellulose, polyquaternium-1, polyquaternium-6, polyquaternium-10, guar, hydroxypropylguar, hydroxypropylmethylguar, cationic guar, carboxymethylguar, hydroxymethylchitosan, hydroxypropylchitosan, carboxymethylchitosan, N-[(2-hydroxy-3-trimethylammonium)propyl]chitosan chloride, water-soluble chitosan, hyaluronic acid and its salts, chondroitin sulfate, heparin, dermatan sulfate, amylose, amylopectin, pectin, locust bean gum, alginate, dextran, carrageenan, xanthan gum, gellan gum, scleroglucan, schizophyllan, gum arabic, gum ghatti, gum karaya, gum tragacanth, pectins, starch and its modifications, tamarind gum, poly(vinyl alcohol), poly(ethylene oxide), poly(ethylene glycol), poly(methyl vinyl ether), polyacrylamide, poly(N,N′-dimethylacrylamide), poly(N-vinylacetamide), poly(N-vinylformamide), poly(2-hydroxyethyl methacrylate), poly(glyceryl methacrylate), poly(N-vinylpyrrolidone), poly(dimethylaminoethyl methacrylate), poly(dimethylaminopropyl acrylamide), polyvinylamine, poly(N-isopropylacrylamide) and poly(N-vinylcaprolactam), the latter two hydrated below their Lower Critical Solution Temperatures, and the like, and combinations thereof.

If anionic hydrophilic polymers are utilized for enhancing viscosity, the overall polymer negative charge may electrostatically attract and accumulate the antibiotics and a greater concentration of antibiotic will then be needed to provide biocidal efficacy comparable to the utilization of a neutral or cationic water-soluble polymer. Thus, preferred water-soluble polymers are neutral or cationic in charge. Examples of neutral water-soluble polymers include, but are not limited to, hydroxyethylcellulose, hydroxypropylcellulose, hydroxypropylmethylcellulose, guar, hydroxypropylguar, hydroxypropylmethylguar, poly(ethylene oxide), and poly(N-vinylpyrrolidone). Examples of cationic water-soluble polymers include, but are not limited to, cationic chitosans, cationic cellulosics, and cationic guar. Chitosan polymers may also enhance the antimicrobial behavior of the hydrolyzed collagen-based composition. More preferred hydrophilic polymers comprise hydroxypropylmethylcellulose, hydroxypropylcellulose, hydroxypropylguar, hydroxymethylchitosan, poly(ethylene oxide), N-[(2-hydroxy-3-trimethylammonium)propyl]chitosan chloride, with hydroxymethylpropylcellulose being most preferred.

Essential oils can also be added to the hydrolyzed collagen-based composition as fragrance or aromatic agents, and/or as antimicrobial agents, including thymol, menthol, sandalwood, camphor, cardamom, cinnamon, jasmine, lavender, geranium, juniper, menthol, pine, lemon, rose, eucalyptus, clove, orange, mint, linalool, spearmint, peppermint, lemongrass, bergamot, citronella, cypress, nutmeg, spruce, tea tree, wintergreen (methyl salicylate), vanilla, and the like. More preferred essential oils include thymol, sandalwood oil, wintergreen oil and eucalyptol for antimicrobial properties and pine oil for fragrance.

The hydrolyzed collagen-based composition can also include wetting agents, buffers, gelling agents or emulsifiers. Other excipients include various water-based buffers ranging in pH from 5.0-7.5, silicones, polyether copolymers, vegetable and plant fats and oils, vitamins, laurate esters, myristate esters, palmitate esters, and stearate esters.

In some embodiments, one or more biologically active agents may be incorporated into the hydrolyzed collagen-based composition to provide a medical benefit to a living host. Examples of biologically active agents that can be incorporated into the hydrolyzed collagen-based composition include, but are not limited to, cells, stem cells, amniotic tissue, amniotic cells, exosomes, growth factors, decellularized extracellular matrix derived from cells, micronized decellularized tissue, granulated crosslinked bovine tendon collagen and glycosaminoglycans, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, topical retinoids, hormones, tumor-specific antibodies, antisense oligonucleotides, small interfering RNA (siRNA), anti-VEGF RNA aptamer, nucleic acids, DNA, DNA fragments, DNA plasmids, Si-RNA, mRNA, transfection agents, vitamins, essential oils, liposomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-tluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle. Such biologically active agents could be in either of the (R)-, (R, S)-, or (S)-configuration, or a combination thereof.

In some embodiments, the hydrolyzed collagen-based composition comprises at least one of cells, stem cells, amniotic tissue, amniotic cells, exosomes, growth factors, decellularized extracellular matrix derived from cells, micronized decellularized tissue, granulated collagen, gelatin, or glycosaminoglycans. In some embodiments, the cells are animal cells. In some embodiments, the cells can be mammalian cells or non-mammalian cells.

The hydrolyzed collagen-based composition may be delivered in a variety of forms. Exemplary forms include, but are not limited to, powders, films, sheets, liquids, creams, foams, lotions, gels, aerosols, tablets and capsules. The hydrolyzed collagen-based composition can also be imbibed by swabs, cloth, sponges, foams, adhesives, wound dressing materials and non-woven and paper products, such as paper towels and wipes. Formulations of the hydrolyzed collagen-based compositions described herein may additionally include organic solvents, emulsifiers, gelling agents, moisturizers, stabilizers, time release agents, dyes, and like components commonly employed in formulations for body administration.

The hydrolyzed collagen-based composition described herein may also be applied to catheters, and other medical devices, in a hydrated or dried form to provide a coating that can prevent microbial attachment to the catheter, or other medical devices, when they are introduced to the body.

Alternatively, the hydrolyzed collagen-based composition described herein can be added to a solid or porous support, such as a polymeric foam, a polymer film, a woven, knitted or nonwoven material, and dried. The treated porous support can then be applied directly to a tissue or medical device. In this case, the porous support may also absorb tissue exudate, creating a hydrated environment for controlled release of hydrolyzed collagen, antibiotic, or other additives.

In another aspect, a method of treating tissue comprising contacting the tissue with any hydrolyzed collagen-based composition described herein is provided.

In some embodiments, the tissue being treated is impaired tissue. In some embodiments, the impaired tissue comprises at least one of a cut, a wound, a lesion, a rash, a fistula, a burn, a void, a surgical site, diabetic foot ulcer, venous ulcer, pressure ulcer, tissue affected by cellulitis, tissue degraded by inflammation, dehisced wounds, necrotic wounds, traumatic wounds with foreign bodies (i.e., puncture wounds), or a medical implant site.

### Experimental

The following materials and abbreviations are used in the experimental section.

| | Source | Trade Name | Lot | Units (mg/mL) |
|---|---|---|---|---|
| Hydrolyzed | Sanara | Cellerate | HY033 | |
| collagen | MedTech | | | |
| Hydrolyzed | Gelita | Peptiplus XB | 8621390-082322 | |
| collagen P | | | | |
| Gelatin 150 | Gelita | | 614901 | |
| Bloom | | | | |
| Limed Bone Gel | | | | |
| Pullulan | BOC | | B21B10272 | |
| Maltodextrin | GPC | | M172089001 | |
| Amphotericin B | Thermofisher | | S06K027 | 0.25 |
| | RPI | | 189079-22171 | n/a |
| Gentamicin/Amphotericin | GIBCO | | 2746432 | 5 |
| 5/0.125 | Thermofisher | | | |
| (mg/mL) | | | | |
| Gentamicin | RPI | | 226162-239648 | |
| Vancomycin | Thermofisher | | X261512 | 50 |
| | RPI | | 248467-258961 | n/a |
| Cefazolin | Thermofisher | Ancef | M09L008 | |
| Sensiva SC 50 | Schülke & Mayr | | 1178933 | |
| Glycerol 1-(2- | | | | |
| ethylhexyl) | | | | |
| ether) | | | | |
| Sensiva SC 10 | Schülke & Mayr | | 1203109 | |
| 1,2- | | | | |
| Dihydroxyoctane | | | | |

The hydrolyzed collagen (Cellerate) is from bovine collagen and comprises collagen type I, II, IV, V, VI, and XVII. In some embodiments, the hydrolyzed collagen is formed from collagen including at least collagen type I. In some embodiments, the hydrolyzed collagen is formed from collagen including at least collagen type II. In some embodiments, the hydrolyzed collagen is formed from collagen including at least collagen type IV. In some embodiments, the hydrolyzed collagen is formed from collagen including at least collagen type V. In some embodiments, the hydrolyzed collagen is formed from collagen including at least collagen type VI. In some embodiments, the hydrolyzed collagen is formed from collagen including at least collagen type XVII.

### Example 1: Hydrolyzed Collagen and Amphotericin B

The effectiveness of hydrolyzed collagen blended with Amphotericin B was evaluated in a standard kill-rate test where the formulations were dissolved in phosphate buffered saline followed by the addition of microbial inoculum. The antibiotic efficacy against planktonic microorganisms was measured after 24 hours, 48 hours and 7 days. The formulations shown in Table 1 were tested for antibiotic efficacy (three replicates per formulation). Complete kill of planktonic gram-negative bacteria (P. aeruginosa), gram-positive bacteria (MRSA), and yeast (C. albicans) was observed at 24 hours with sustained effectiveness throughout the 7-day testing period as no regrowth was observed.

TABLE 1
Hydrolyzed collagen with Amphotericin B (1:1 weight ratio)

| | | treatment | log |
|---|---|---|---|
| microbe | AmphotericinB + HC | time | reduction |
| MRSA | 2 + 2 mg/mL | 24 h | 6.24 |
| P. | 2 + 2 mg/mL | 24 h | 6.25 |
| aeruginosa | | | |
| C. albicans | 0.8 + 0.8 mg/mL | 24 h | 6.13 |

### Example 2: Hydrolyzed Collagen and Gentamicin/Amphotericin

The effectiveness of hydrolyzed collagen blended with Gentamicin (Gentamicin/Amphotericin) was evaluated in a standard kill-rate test where the formulations were dissolved in phosphate buffered saline followed by the addition of microbial inoculum. The antibiotic efficacy against planktonic microorganisms was evaluated after 24 hours, 48 hours and 7 days. The formulations shown in Table 2 were tested for antibiotic efficacy (three replicates per formulation). Complete kill of planktonic gram-negative bacteria (P. aeruginosa), gram-positive bacteria (MRSA), and yeast (C. albicans) was observed at 24 hours with sustained effectiveness throughout the 7-day testing period as no regrowth was observed.

TABLE 2
Hydrolyzed collagen with Gentamicin/Amphotericin/HC

| | | | treatment | log |
|---|---|---|---|---|
| microbe | Gentamicin + Amphotericin + HC | | time | reduction |
| MRSA | 0.5 + 0.0125 + 0.5 | mg/mL | 24 h | 6.15 |
| P. | 0.5 + 0.0125 + 0.5 | mg/mL | 24 h | 6.27 |
| aeruginosa | | | | |
| C. albicans | 2 + 0.05 + 2 | mg/mL | 24 h | 6.27 |

### Example 3: Hydrolyzed Collagen with Vancomycin

The effectiveness of hydrolyzed collagen blended with Vancomycin was evaluated in a standard kill-rate test where the formulations were dissolved in phosphate buffered saline followed by the addition of microbial inoculum. The antibiotic efficacy against planktonic microorganisms was evaluated after 24 hours, 48 hours and 7 days. The formulations shown in Table 3 below were tested for antibiotic efficacy (three replicates per formulation). Complete kill of planktonic gram-negative bacteria (P. aeruginosa), gram-positive bacteria (MRSA), and yeast (C. albicans) was observed at 24 hours with sustained effectiveness throughout the 7-day testing period as no regrowth was observed (Table 3).

TABLE 3
Hydrolyzed collagen with Vancomycin (1:1 weight ratio)

| | | treatment | log |
|---|---|---|---|
| microbe | Vancomycin + HC | time | reduction |
| MRSA | 1 + 1 mg/mL | 24 h | 6.18 |
| P. aeruginosa | 1 + 1 mg/mL | 24 h | 6.22 |
| C. albicans | 2 + 2 mg/mL | 24 h | 6.21 |

### Example 4: Hydrolyzed Collagen with Cefazolin

The effectiveness of hydrolyzed collagen blended with Cefazolin was evaluated in a standard kill-rate test where the formulations were dissolved in phosphate buffered saline followed by the addition of microbial inoculum. The antibiotic efficacy against planktonic microorganisms was evaluated after 24 hours. The formulations shown in Table 4, below, were tested for antibiotic efficacy (three replicates per formulation). Less than 1 log reduction of planktonic gram-negative bacteria (P. aeruginosa), gram-positive bacteria (MRSA), and yeast (C. albicans) was observed at 24 hours.

TABLE 4
Hydrolyzed collagen with Cefazolin

| | | treatment | log |
|---|---|---|---|
| microbe | Cefazolin + HC | time | reduction |
| MRSA | 0.05 + 0.05 mg/mL | 24 h | 0.71 |
| P. aeruginosa | 0.05 + 0.05 mg/mL | 24 h | 0.67 |
| C. albicans | 0.05 + 0.05 mg/mL | 24 h | 0.62 |

### Example 5: Hydrolyzed Collagen with Amphotericin B, Gentamicin, Vancomycin, and Cefazolin

The effectiveness of hydrolyzed collagen blended with Amphotericin B, Gentamicin, Vancomycin, and Cefazolin was evaluated in a standard kill rate test where the dry test materials (0.5 g or 1 g total) were dissolved in phosphate buffered saline (5 or 10 mL, respectively) followed by addition of P. aeruginosa microbial inoculum (0.1 mL) and incubated at 25° C. for 24 hours. 1 mL of this mixture was then added to 9 mL of Dey Engley (DE) buffer and vortexed. After vortexing, 0.1 mL was plated onto agar plates (three replicates) and incubated at 37° C. The antibiotic efficacy against planktonic gram-negative bacteria (P. aeruginosa) after 4 hours and 24 hours was determined by enumeration of colonies. The antibiotic formulations shown in Table 5 below were tested for antibiotic efficacy and in general demonstrated improved log reduction with increasing antibiotic concentration.

TABLE 5
Hydrolyzed collagen with Amphotericin B, Gentamicin, Vancomycin
and Cefazolin: P. aeruginosa log reduction

| | | | | | 4 hour | 24 hour |
|---|---|---|---|---|---|---|
| | | | | | treatment | treatment |
| Test materials by weight percent solids | | | | | log | log |
| Amphotericin | Gentamicin | Vancomycin | Cefazolin | HC | reduction | reduction |
| 0 wt % | 0 wt % | 0 wt % | 0 wt % | 100 wt % | 0 | 0 |
| 0.025 | 1.00 | 1.02 | 0 | 97.955 | 0.72 | 4.07 |
| 1.025 | 1.00 | 1.00 | 0 | 96.975 | 0.97 | 3.73 |
| 0.01 | 0.01 | 0.01 | 0 | 99.97 | 0.48 | 2.03 |
| 0.001 | 0.001 | 0.001 | 0 | 99.997 | 0.46 | 1.55 |
| 20.07 | 19.99 | 19.91 | 19.97 | 20.05 | — | 6.22 |

### Example 6: Hydrolyzed Collagen with Antibiotics and Humectants

Hydrolyzed collagen powder was blended with antibiotics (liquid or powder) and liquid humectants—vicinal diols SC50 and SC10 as shown in Table 6, Formulation A. Formulation A was also tested for efficacy against gram-positive bacteria (MRSA), gram-negative bacteria (P. aeruginosa), and yeast (C. albicans) as described above, see Table 7, and resulted in complete eradication of all three microbes within 24 h test time.

TABLE 6
Composition of hydrolyzed collagen with antibiotics and vicinal diols

| | Test materials by weight percent solids | | | | | |
|---|---|---|---|---|---|---|
| | Amphotericin | Gentamicin | Vancomycin | SC 50 | SC 10 | HC |
| Formulation | 1.00 | 1.00 | 1.01 | 0.30 | 0.10 | 96.59 |
| A | | | | | | |

TABLE 7
Hydrolyzed collagen with antibiotics and vicinal
diols: activity against planktonic microorganisms

| | Formulation | treatment | log |
|---|---|---|---|
| microbe | A | time | reduction |
| MRSA | mg/mL | 24 h | 6.25 |
| P. aeruginosa | mg/mL | 24 h | 6.22 |
| C. albicans | mg/mL | 24 h | 6.18 |

### Example 8: Hydrolyzed Collagen with Antibiotics and with Protein and Polysaccharides

Hydrolyzed collagen powder was blended with antibiotics (liquid or powder) and other additive powders to yield blended powder (Table 8). All of the samples were freely flowable and non-tacky.

TABLE 8
Hydrolyzed collagen with antibiotics

| | | wt % | |
|---|---|---|---|
| Chemical | Grams | Solids | Observations |
| Hydrolyzed collagen P | 2.5003 | 49.9948 | Color = light yellow |
| Gelatin | 2.5008 | 50.0047 | Freely flowable, non-cohesive, similar angle of |
| Amphotericin B | 0.0257 mg | 0.0005 | repose, can be compressed/tapped to occupy |
| | | | slightly smaller volume. |
| Hydrolyzed collagen P | 4.5002 | 89.98 | Color = light yellow |
| Gelatin | 0.5006 | 10.01 | Freely flowable, non-cohesive, similar angle of |
| Gentamicin/Amphotericin | 0.5095 mg | 0.1 | repose, can be compressed/tapped to occupy |
| | | | slightly smaller volume. |
| Hydrolyzed collagen | 2.5005 | 49.9927 | Color = light yellow |
| Gelatin | 2.4887 | 49.7568 | Freely flowable, non-cohesive, similar angle of |
| Vancomycin | 12.53 mg | 0.2505 | repose, can be compressed/tapped to occupy |
| | | | slightly smaller volume. |
| Hydrolyzed collagen | 3.7502 | 74.9866 | Color = light yellow |
| Pullulan | 1.2509 | 25.0122 | Freely flowable, non-cohesive, similar angle of |
| Amphotericin B | 0.0631 mg | 0.0012 | repose, can be compressed/tapped to occupy |
| | | | slightly smaller volume. |
| Hydrolyzed collagen | 1.2507 | 25.0167 | Color = light yellow |
| Maltodextrin | 3.7475 | 74.9582 | Freely flowable, non-cohesive, similar angle of |
| Gentamicin/Amphotericin | 1.255 mg | 0.0251 | repose, can be compressed/tapped to occupy |
| | | | slightly smaller volume. |
| Hydrolyzed collagen | 1.4031 | 70.0035 | Color = light yellow |
| Gelatin | 0.5986 | 29.8662 | Freely flowable, non-cohesive, similar angle of |
| Gentamicin | 0.063 mg | 0.0051 | repose, can be compressed/tapped to occupy |
| Vancomycin | 2.5 mg | 0.1252 | slightly smaller volume. |
| Hydrolyzed collagen | 1.2608 | 62.3537 | Color = light yellow |
| Gelatin | 0.5085 | 25.1491 | Freely flowable, non-cohesive, similar angle of |
| Pullulan | 0.2501 | 12.37 | repose, can be compressed/tapped to occupy |
| Vancomycin | 2.5 mg | 0.1266 | slightly smaller volume. |
| Amphotericin B | 0.012 mg | 0.0006 | |

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents

What is claimed is:

1. A hydrolyzed collagen-based composition comprising:
25 wt % to 99.9995 wt % of hydrolyzed collagen; and
0.0005 wt % to 50 wt % of an antibiotic component, wherein the percentages are based on the percent solids of the composition.

2. The hydrolyzed collagen-base composition of claim 1, wherein the antibiotic component comprises at least one of a polyene antimycotic, an aminoglycoside antibiotic, a glycopeptide antibiotic, a beta-lactam antibiotic or a derivative thereof.

3. The hydrolyzed collagen-based composition of claim 1, comprising at least 50 wt % of hydrolyzed collagen, based on the percent solids of the composition.

4. The hydrolyzed collagen-based composition of claim 1, comprising at least 0.005 wt % of the antibiotic component, based on the percent solids of the composition.

5. The hydrolyzed collagen-based composition of claim 1, wherein the antibiotic component comprises at least two antibiotics independently selected from polyene antimycotics, aminoglycoside antibiotics, beta-lactam antibiotics, and glycopeptide antibiotics.

6. The hydrolyzed collagen-based composition of claim 5, wherein a ratio between any two components of the antibiotic component that are present is in a range from 1:200 to 200:1, based on weight of the components.

7. The hydrolyzed collagen-based composition of claim 1, wherein the antibiotic component comprises at least three antibiotics, each of which is independently selected from polyene antimycotics, aminoglycoside antibiotics, beta-lactam antibiotics, and glycopeptide antibiotics.

8. The hydrolyzed collagen-based composition of claim 1, wherein the antibiotic component comprises at least four antibiotics, each of which is independently selected from polyene antimycotics, aminoglycoside antibiotics, beta-lactam antibiotics, and glycopeptide antibiotics.

9. The hydrolyzed collagen-based composition of claim 1, wherein the antibiotic component comprises at least one antibiotic from each of the following classes of antibiotics: polyene antimycotics, aminoglycoside antibiotics, beta-lactam antibiotics, and glycopeptide antibiotics.

10. The hydrolyzed collagen-based composition of claim 1, wherein the composition exhibits a kill of at least 1 log reduction of planktonic microbes.

11. The hydrolyzed collagen-based composition of claim 1, further comprising a hydrophobic vicinal diol selected from the group consisting of monoalkyl glycol, glycerol alkyl ether, monoacyl glycerol, and combinations thereof.

12. The hydrolyzed collagen-based composition of claim 11, comprising 0.05 to 20 wt % of the hydrophobic vicinal diol, based on the percent solids of the composition.

13. The hydrolyzed collage-based composition of claim 1, further comprising 0.01 wt % to 5 wt % of a chelating agent, based on the percent solids of the composition.

14. The hydrolyzed collagen-based composition of claim 1, further comprising a biologically active agent selected from cells, stem cells, amniotic tissue, amniotic cells, exosomes, growth factors, decellularized extracellular matrix derived from cells, micronized decellularized tissue, granulated crosslinked bovine tendon collagen and glycosaminoglycans, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, topical retinoids, hormones, tumor-specific antibodies, antisense oligonucleotides, small interfering RNA (siRNA), anti-VEGF RNA aptamer, nucleic acids, DNA, DNA fragments, DNA plasmids, Si-RNA, mRNA, transfection agents, vitamins, essential oils, liposomes, exosomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-tluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle.

15. The hydrolyzed collagen-based composition of claim 1, further comprising at least one of cells, stem cells, amniotic tissue, amniotic cells, exosomes, growth factors, micronized decellularized tissue, decellularized extracellular matrix derived from cells, granulated collagen, gelatin, or glycosaminoglycans.

16. The hydrolyzed collagen-based composition of claim 1, further comprising at least one additional ingredient selected from glycolipids, glycoproteins, immunological response modifiers, saccharides, steroids, and polysaccharides.

17. The hydrolyzed collagen-based composition of claim 1, wherein the composition has a form selected from a powder, a liquid, a gel, a paste, a cream, a suspension, an emulsion, a film, a sheet, a foam, a lotion, a spray, an aerosol, a capsule, or a tablet.

18. The hydrolyzed collagen-based composition of claim 1, wherein the composition comprises a fluid selected from the group consisting of water, isotonic saline, balanced salt solution, buffer solution, Ringer's solution, cell culture media, stem cell media, serum, plasma, amniotic fluid, Wharton's jelly, nutrient broth, antiseptic solutions, and combinations thereof.

19. A method of treating tissue comprising contacting the tissue with a hydrolyzed collagen-based composition of claim 1.

20. The method of claim 19, wherein the hydrolyzed collagen-based composition comprises an antibiotic component comprising Amphotericin B, Gentamicin, Vancomycin, Cephalosporin, derivatives thereof, or combinations thereof.

21. The method of claim 19, wherein the hydrolyzed collagen-based composition exhibits a kill of at least 1 log reduction of planktonic microbes.

22. The method of claim 19, wherein the hydrolyzed collagen-based composition is applied topically, via injection, or via ingestion.
